(19) United States
(12) Patent Application Publication
(10) Pub. No.: US 2026/0183504 A1
(43) Pub. Date: Jul. 2, 2026

(54) SWIVEL CONNECTOR FOR BREATHING TUBE ASSEMBLY

(71) Applicant: FISHER & PAYKEL HEALTHCARE LIMITED, Auckland (NZ)

(72) Inventors: Paul Mathew FREESTONE, Auckland (NZ); Xueyan BIAN, Auckland (NZ); Christopher Jaye Norman AMADIO, Auckland (NZ); Craig Robert PRENTICE, Auckland (NZ); Simon MITTERMEIER, Auckland (NZ); Andrew SHARP, Auckland (NZ); Sachit Rahil JOHN, Auckland (NZ)

(21) Appl. No.: 19/131,475

(22) PCT Filed: Nov. 21, 2023
(86) PCT No.: PCT/NZ2023/050135

§ 371 Date: May 20, 2025

(30) Foreign Application Priority Data
Nov. 21, 2022 (NZ) 794679

(51) Int. Cl. A61M 16/08; A61M 16/06

(52) U.S. Cl. CPC A61M 16/0816; A61M 16/06

(57) ABSTRACT

A swivel connector for a gas delivery tube is provided. The swivel connector comprises a first conduit component; and a second conduit component configured to receive the first conduit component. The first conduit component comprises a proximal end configured to be connected to the gas delivery tube, and a distal end. The first conduit component and the second conduit component are configured to be connected together so as to be rotatable relative to one another when connected together. Swivel connectors are provided in which the first and second conduit components are permanently connected together. Various connection features are disclosed which provide this permanent connection. Swivel connectors are disclosed in which one conduit component is substantially entirely received within the other, such that one component cannot be easily gripped by a user.

### FIELD OF THE DISCLOSURE

This disclosure generally relates to a swivel connector for a breathing tube assembly, for use with, or comprising part of, a respiratory therapy system. More particularly, the disclosure relates to a swivel connector that connects a gas delivery tube to a patient interface. This application claims priority from New Zealand provisional patent application 794679, the entire contents of which are hereby incorporated by reference.

### BACKGROUND

Respiratory therapy systems using breathing tubes to provide gases to a patient during respiratory therapy are known in the art. Such systems can be used for Nasal High Flow (NHF) therapy, and the treatment of obstructive sleep apnoea (OSA) by continuous positive airway pressure (CPAP). Such respiratory therapy systems deliver respiratory gases to the patient via a breathing tube and a patient interface (for example, a mask or cannula). The delivery of respiratory gases from the flow generator and/or humidification apparatus to the patient interface is via one or more gas delivery tubes, known as breathing tubes, arranged in series.

Respiratory therapy systems in the prior art may deliver respiratory gases to the patient via multiple breathing tubes to form a continuous pathway between the source of respiratory gases and the patient interface. Typically, the source of respiratory gases, for example, a flow generator such as a blower, is coupled to a larger breathing tube, and the patient interface is coupled to a shorter, smaller and/or lightweight connecting tube. A connector enables a connection between the two tubes and establish a gases flow path. Typically, the end of a breathing tube has proprietary connectors or intermediate coupling components that facilitate connection and form a continuous lumen.

During therapy, the user may use the breathing tubes and patient interface in various positions and locations. For example, the user may wish to rotate and move their head, requiring movement between the breathing tube(s) and patient interface. This may lead to twisting and/or tangling of the breathing tubes, which can affect flexibility and/or restrict freedom of movement of the user. The usability of the patient interface can be reduced and cause discomfort for the user. In some cases, this may lead to kinks in the breathing tube resulting in a flow restriction, and/or damage to the breathing tube.

Respiratory therapy systems sometimes comprise a swivel connector along the gases flow path, for example, between the breathing tube and the connecting tube. A swivel connector allows the breathing tube and connecting tube to rotate relative to each other.

A swivel connector is described that at least partially overcomes or ameliorates at least one disadvantage of the prior art, or at least provides a useful choice.

### SUMMARY OF THE DISCLOSURE

According to an aspect of this disclosure there is provided a swivel connector for a gas delivery tube, the swivel connector comprising
a first conduit component; and
a second conduit component configured to receive the first conduit component;
wherein the first conduit component comprises a proximal end configured to be connected to the gas delivery tube, and a distal end;
wherein the first conduit component and the second conduit component are configured to be connected together;
wherein the first conduit component and second conduit component are configured to be rotatable relative to one another when connected together.

An exterior surface of the first conduit component may be adjacent an interior surface of the second conduit component.

The first conduit component may comprise a proximal opening at the proximal end, and a distal opening at the distal end, a wall extending between the openings in a direction of a longitudinal axis of the first conduit component. The wall may comprise an exterior surface of the first conduit component, extending in a direction of a longitudinal axis of the first conduit component.

The proximal opening may comprise an inlet to a gases flow path through the first conduit component.

The distal opening may comprise an outlet of the gases flow path through the first conduit component.

The second conduit component may comprise a proximal end and a distal end. The second conduit component may comprise a proximal opening at the proximal end, and a distal opening at the distal end, a wall extending between the inlet and the outlet in a direction of a longitudinal axis of the second conduit component. The wall may comprise an interior surface of the second conduit component, extending in a direction of a longitudinal axis of the second conduit component.

The exterior surface may be substantially aligned with the interior surface. The exterior surface may be parallel with the interior surface. One surface may be inclined relative to the other surface.

The first conduit component and second conduit component may be arranged coaxially.

The first conduit component and second conduit component may be connected permanently.

The first conduit component and second conduit component may be connected with an interference fit.

The first conduit component and second conduit component may be connected by a snap-fit mechanism.

The first conduit component may comprise at least one engagement formation configured to engage the second conduit component to provide the snap-fit mechanism.

The engagement formation may engage the interior surface of the second conduit component.

The engagement formation may be a protrusion.

The engagement formation may be located on the distal end of the first conduit component.

The engagement formation may project radially outwardly from an exterior surface of the first conduit component.

The distal end of the first conduit component may further comprise an undercut.

The undercut may be located closer to the proximal end of the first conduit component relative to the protrusion.

The first conduit component may further comprise at least one flexing arm extending longitudinally from the distal end. The flexing arm may be configured to be able to resiliently move relative to the remainder of the first conduit component. The flexing arm may be configured to be able to flex from a rest position to a flexed position under application of a force, and to move back to the rest position on removal of the force.

The engagement formation may be located on the flexing arm.

The first conduit component may have a plurality of flexing arms.

At least two of the plurality of flexing arms may be located on diametrically opposite sides of the distal end of the first conduit component.

The interior surface of the second conduit component may comprise at least one radially extending protrusion configured to engage with the at least one engagement formation of the first conduit component.

The at least one radially extending protrusion may be a ledge.

The ledge may extend peripherally around the interior surface of the second conduit component.

When assembled, the ledge is configured to provide a bearing surface for the protrusion of the first conduit component.

The engagement formation of the first conduit component may be a recess that extends at least partially peripherally around the exterior surface of the first conduit component that engages a protrusion on the interior surface of the second conduit component.

The second conduit component may be configured to connect to a second gas delivery tube. For example, the second gas delivery tube may be a connecting tube, connecting the swivel connector (and therefore the gas delivery tube) to a patient interface.

The second conduit component may removably connect to the second gas delivery tube via a friction fit.

The second conduit component may be configured to engage an end portion of the second gas delivery tube.

The end portion of the gas delivery tube may have an intermediate coupling component.

The first conduit component may be configured to be coupled to the gas delivery tube via overmoulding.

The first conduit component and second conduit component may be configured so that a clearance between the exterior surface of the first conduit component and the internal surface of the second conduit component is provided, when the first conduit component and second conduit component are connected together, the clearance allowing for relative rotation between the first conduit component and second conduit component, while minimising leakage of gases from the swivel connector.

The first conduit component may have a length between the proximal end and distal end along a longitudinal axis, wherein the first conduit component may be configured to be connected to the second conduit component such that at least 25% of the length of the first conduit component is received by, that is, is inside, the second conduit component.

The second conduit component may have a length between the proximal end and distal end along a longitudinal axis, wherein when connected, the first conduit component overlaps approximately 40% of the length of the second conduit component.

When connected, at least one portion of the proximal end of the first conduit component is covered by the second conduit component such that it cannot be gripped by a user. Any part of the proximal end of the first conduit component that is exposed, may be insufficient to be gripped by a user, or at least may be insufficient to enable a user to apply sufficient force to the proximal end to damage and/or break the connection between the first and second conduit components.

When assembled, at least another portion of the proximal end of the first conduit component may be exposed.

The length of the exposed portion of the proximal end of the first conduit component may be less than approximately 10 mm, in some embodiments, the exposed portion is approximately 6 mm.

The proximal end of the first conduit component and a proximal end of the second conduit component may be arranged such that they are approximately co-planar, when the first and second conduit components are connected together.

The first conduit component may comprise at least one shoulder extending outwardly away from a longitudinal axis of the first conduit component, the shoulder extending at least partially around the perimeter of the exterior surface of the first conduit component.

The shoulder may be disposed towards the proximal end of the first conduit component.

The shoulder may be configured to abut a proximal end of the second conduit component at the proximal end of the first conduit component.

The shoulder of the first conduit component may be configured to abut a shoulder of the second conduit component.

The first conduit component and second conduit component may each be generally cylindrical.

According to a further aspect of this disclosure there is provided a swivel connector for a gas delivery tube, the swivel connector comprising
a first conduit component; and
a second conduit component configured to receive the first conduit component;
wherein the first conduit component comprises a proximal end configured to be connected to the gas delivery tube, and a distal end;
wherein the first conduit component and second conduit component are configured to be permanently connected together, and to be rotatable relative to one another, when connected together.

According to a further aspect of this disclosure there is provided a swivel connector for a gas delivery tube, the swivel connector comprising
a first conduit component having a first end and a second end, the second end being configured to be connected to the gas delivery tube; and
a second conduit component having a first end and a second end;
wherein the first conduit component and the second conduit component are configured to be connected together; wherein the first end of the first conduit is configured to be received by the first end of the second conduit component such that an interior surface of the second conduit component is adjacent an exterior surface of the first conduit component.

The first conduit component may comprise a first opening at the first end, and a second opening at the second end, a wall extending between the openings in a direction of a longitudinal axis of the first conduit component. The wall may comprise an exterior surface of the first conduit component, extending in a direction of a longitudinal axis of the first conduit component.

The first opening may comprise an inlet to a gases flow path through the first conduit component.

The second opening may comprise an outlet of the gases flow path through the first conduit component.

The second conduit component may comprise a first end and a second end. The second conduit component may comprise a first opening at the first end, and a second opening at the second end, a wall extending between the inlet and the outlet in a direction of a longitudinal axis of the second conduit component. The wall may comprise an interior surface of the second conduit component, extending in a direction of a longitudinal axis of the second conduit component.

The exterior surface may be substantially aligned with the interior surface. The exterior surface may be parallel with the interior surface. One surface may be inclined relative to the other surface.

According to an aspect of this disclosure there is provided a swivel connector for a gas delivery tube, the swivel connector comprising:
a first conduit component and a second conduit component, the first conduit component configured to be connected to the gas delivery tube,
wherein the first conduit component is configured to be connected to the second conduit component such that the first conduit component is received in the second conduit component sufficiently that the first conduit component cannot be gripped by a user.

Any part of the first conduit component that is exposed, may be insufficient to be gripped by a user, or at least may be insufficient to enable a user to grip the first conduit component with sufficient force to the first conduit component to damage and/or break the connection between the first and second conduit components.

According to an aspect of this disclosure there is provided a swivel connector for a gas delivery tube, the swivel connector comprising:
a first conduit component having a proximal end and a distal end;
a second conduit component configured to receive the first conduit component;
wherein the first conduit component and the second conduit component are configured to be connected together;
the first conduit component comprising a shoulder disposed towards the proximal end of the first conduit component;
wherein, when the first component is connected to the second component, a portion between the shoulder and distal end of the first conduit component is covered by the second conduit component, and a portion between the proximal end and the shoulder of the first conduit component is exposed.

The portion between the shoulder and distal end of the first conduit component may be enclosed by the second conduit component, such that this portion is covered by the first conduit component.

The portion between the shoulder and distal end of the first conduit component may have a first length along a longitudinal axis and the portion between the shoulder and proximal end of the first conduit component may have a second length along a longitudinal axis, wherein the first length is more than the second length, in some embodiments the first length is more than 1.5 times the second length, in some embodiments the first length is more than two times the second length.

According to another aspect of this disclosure there is provided a swivel connector for a gas delivery tube comprising
a first conduit component having a first end configured to be connected to the gas delivery tube, and a second end; and
a second conduit component comprising an opening to receive the first conduit component;
wherein the first conduit component and the second conduit component are configured to be connected together;
the first conduit component being configured to be received in the second conduit component such that the first end of the first conduit component and the first end of the second conduit component are co-planar, when the first and second conduit components are connected together.

The second conduit component may comprise a first end, and a second end, the second end being configured to be connected to a gas delivery tube.

The opening may be located at the first end of the second conduit connector.

The first conduit component may comprise a shoulder extending at least partially around the periphery of the first conduit component.

The shoulder may be located at the first end of the first conduit component.

The shoulder may be configured to abut the first end of the second conduit component.

The first conduit component may comprise a second shoulder located part way along an axial length of the first conduit component, the second shoulder being configured to abut the first end of the second conduit component.

The second conduit component may comprise a shoulder extending away from a longitudinal axis of the second conduit component, the shoulder extending at least partially around a periphery of the second conduit component.

The shoulder may be disposed towards the first end of the second conduit component.

The shoulder of the second conduit component may extend perpendicularly from an exterior surface of the second conduit component.

The shoulder of the second conduit component may be L-shaped in cross-section, such that the shoulder comprises a perpendicularly extending portion and an axially extending portion.

The shoulder of the first conduit component may comprise a curved wall, when viewed in cross section, the axially extending portion of the shoulder of the second conduit component covering the curved wall.

The shoulder of the second conduit component may be configured to be gripped by a user.

According to another aspect of this disclosure there is provided a swivel connector for a gas delivery tube, the swivel connector comprising:
a male conduit component having a first end configured to connect to the gas delivery tube; and
a female conduit component having a first end,
wherein the male conduit component and the female conduit component are configured to be connected together when the male conduit component is received in the female conduit component;
wherein the male conduit component comprises an end stop protruding from the male conduit component; wherein
the female conduit component abuts the end stop when the male conduit component is received in the female conduit component,
so as to limit axial movement of the female conduit component relative to the male conduit component.

The end stop may be provided at the first end of the male conduit component.

The end stop may protrude from an external surface of the male conduit component.

According to another aspect of this disclosure there is provided a swivel connector for a gas delivery tube, the swivel connector comprising:
a male conduit component and a female conduit component, the male conduit component being configured to be received by the female conduit component such that the male conduit component and female conduit component are coaxial;
the male conduit component having a first end configured to connect to the gas delivery tube, the female conduit component having a first end;
wherein the male conduit component and the female conduit component are configured to be connected together;
the male conduit component having an external surface configured to be adjacent an internal surface of the female conduit component when the conduit components are connected together;
wherein a flange projects radially outwardly from the external surface of the first end of the male conduit component or from an external surface of the first end of the female conduit component, such that the user is able to grip the flange during use.

The male conduit component may comprise a first opening at the first end, and a second opening at the second end, a wall extending between the openings in a direction of a longitudinal axis of the male conduit component. The wall may comprise an exterior surface of the male conduit component, extending in a direction of a longitudinal axis of the male conduit component.

The first opening may comprise an inlet to a gases flow path through the male conduit component.

The second opening may comprise an outlet of the gases flow path through the male conduit component.

The female conduit component may comprise a first end and a second end. The female conduit component may comprise a first opening at the first end, and a second opening at the second end, a wall extending between the inlet and the outlet in a direction of a longitudinal axis of the female conduit component. The wall may comprise an interior surface of the female conduit component, extending in a direction of a longitudinal axis of the female conduit component.

The exterior surface may be substantially aligned with the interior surface. The exterior surface may be parallel with the interior surface. One surface may be inclined relative to the other surface.

According to an aspect of this disclosure there is provided a swivel connector for a gas delivery tube, the swivel connector comprising:
a first conduit component comprising a first end configured to connect to the gas delivery tube; and
a second conduit component configured to be received by the first conduit component;
wherein the first conduit component and the second conduit component are configured to be connected together;
a gripping region extending around a periphery of a portion of an outer surface of the second conduit component;
wherein the gripping region comprises a grip ring configured to be gripped by the user.

According to an aspect of this disclosure there is provided a swivel connector for a gas delivery tube, the swivel connector comprising:
a first conduit component comprising a proximal end configured to connect to the gas delivery tube; and
a second conduit component configured to be received by the first conduit component;
wherein the first conduit component and the second conduit component are configured to be connected together;
wherein the proximal end of the first conduit component further comprises an offset portion.

According to an aspect of this disclosure there is provided a swivel connector for a gas delivery tube, the swivel connector comprising:
a first conduit component comprising a proximal end configured to connect to the gas delivery tube; and
a second conduit component configured to be received by the first conduit component;
wherein the first conduit component and the second conduit component are configured to be connected together;
wherein the proximal end of the first conduit component further comprises an edge that delimits the first conduit component along a longitudinal axis, and the edge comprises an offset portion, the offset portion being configured to allow a portion of the edge to follow a helical orientation of part of the gas delivery tube.

The proximal end of the first conduit component may be overmoulded to an end of the gas delivery tube, such that the proximal end surrounds a portion of the gas delivery tube.

For example, the helical orientation of the edge may follow a helical bead of the gas delivery tube.

According to an aspect of this disclosure there is provided a frame assembly comprising:
a. a frame;
b. a gas delivery tube connected to the frame; and
c. a swivel connector according to any one or more of the above statements.

According to an aspect of this disclosure there is provided a patient interface comprising:
a. a cushion; and
b. a frame, the cushion being mounted on the frame;
c. the frame being connected to a gas delivery tube with a swivel connector according to any one or more of the above statements.

The mask assembly may further comprise a headgear assembly.

According to an aspect of this disclosure there is provided a patient interface assembly comprising:
a. a patient interface;
b. a gas delivery tube configured to be connected to the patient interface; and
c. a swivel connector according to any one or more of the above statements.

According to an aspect of this disclosure there is provided a tube kit comprising:
a. A first gas delivery tube;
b. a second gas delivery tube; and
c. a swivel connector according to any one or more of the above statements.

According to an aspect of this disclosure there is provided a respiratory therapy system comprising:
a. a swivel connector according to any one or more of the above statements; and any one or more of:
b. a patient interface;
c. a gas delivery tube;
d. a humidifier;
e. a flow generator.

### BRIEF DESCRIPTION OF THE DRAWINGS

Embodiments are described with reference to the accompanying drawings, by way of example and without intending to be limiting, in which:

FIG. 1 is a schematic view of a respiratory system according to the present disclosure.

FIG. 2 is a side view of a swivel connector used with part of the respiratory system of FIG. 1.

FIG. 3 is a perspective view of a patient interface assembly in accordance with this disclosure, including a swivel connector.

FIG. 4 is an exploded perspective view of a swivel connector in accordance with the present disclosure, the swivel connector connected to one end of a gas delivery tube.

FIG. 5 is a perspective view of the swivel connector of FIG. 4, in accordance with the present disclosure, the swivel connector connected to one end of a gas delivery tube.

FIG. 6 is a cross-sectional view of the swivel connector as illustrated in FIGS. 4 and 5, with the gas delivery tube omitted for clarity, taken on line A-A of FIG. 4.

FIG. 7 is an enlarged cross-sectional view of part of the swivel connector of FIG. 6.

FIG. 8 is an enlarged cross-sectional view of the part of the swivel connector shown in the circle of FIG. 7.

FIG. 9 is a cross-sectional view of another swivel connector in accordance with the present disclosure.

FIG. 10 is a cross-sectional view of another swivel connector in accordance with the present disclosure.

FIG. 11 is a cross-sectional view of another swivel connector in accordance with the present disclosure.

FIG. 12 is a cross-sectional view of another swivel connector in accordance with the present disclosure.

FIG. 13 is a cross-sectional view of another swivel connector in accordance with the present disclosure, taken along a plane extending through the longitudinal axis of the swivel connector.

FIG. 14 is a cross sectional view of the swivel connector of FIG. 13, taken along a plane that is 90° to the plane used in FIG. 13.

FIG. 15 is a perspective view of a swivel connector according to an embodiment of the present disclosure, the swivel connector comprising a grip ring.

FIG. 16 is another perspective view of the swivel connector of FIG. 15.

FIG. 17 is a side view of the swivel connector of FIG. 15.

FIG. 18 is an exploded view of the swivel connector of FIG. 13.

FIG. 19 is another exploded view of the swivel connector of FIG. 13.

FIG. 20 is a cross-sectional view of an external component of the swivel connector of FIG. 15.

FIG. 21 is a side view of an internal component of the swivel connector of FIG. 20, showing an offset portion at the proximal end.

FIG. 22 is a perspective view of the internal component of the swivel connector of FIG. 21.

23 is a perspective view of an internal component of another embodiment of a swivel connector in accordance with this disclosure, the internal component having an offset portion with a continuous curve.

FIG. 24 is a perspective view of the internal component of the swivel connector of FIG. 21.

FIG. 25 is a perspective view of another swivel connector in accordance with this disclosure.

FIG. 26 is an exploded perspective view of the swivel connector of FIG. 25.

### DETAILED DESCRIPTION

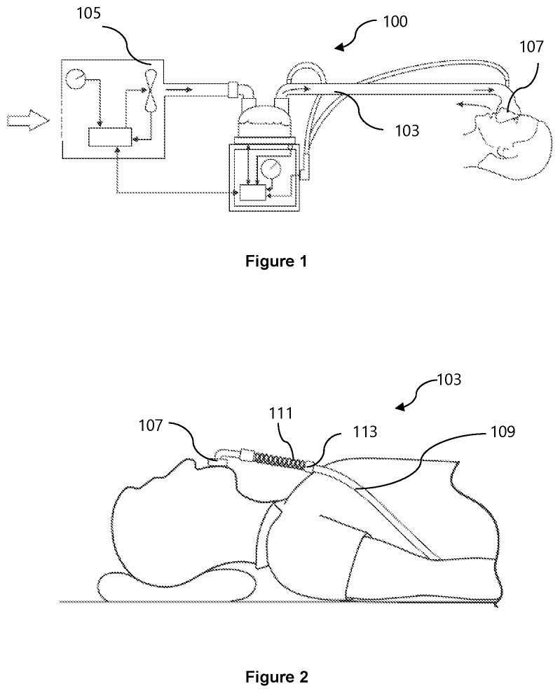

With reference to FIG. 1, a non-limiting example of a typical respiratory therapy system 100 comprises a breathing tube assembly 103 that transports the gases from a gas source 105, such as a CPAP device, to a patient via a patient interface 107.

As illustrated in FIG. 2, the breathing tube assembly 103 is formed in this example by first and second gas delivery tubes 111, 109. A first gas delivery tube 111, hereinafter referred to as the connecting tube 111, extends between the patient interface and the second gas delivery tube 109. The second gas delivery tube 109 is connected to the gas source, and hereinafter is referred to as a breathing tube 109.

The patient interface 107 may, for example be a non-sealing nasal cannula or a face seal comprising a cushion that seals with the patient's face. The seal may be full face, full face over nose, full face under nose, nasal or oral. The patient interface 107 may also comprise, or be configured to be connected to, a headgear system.

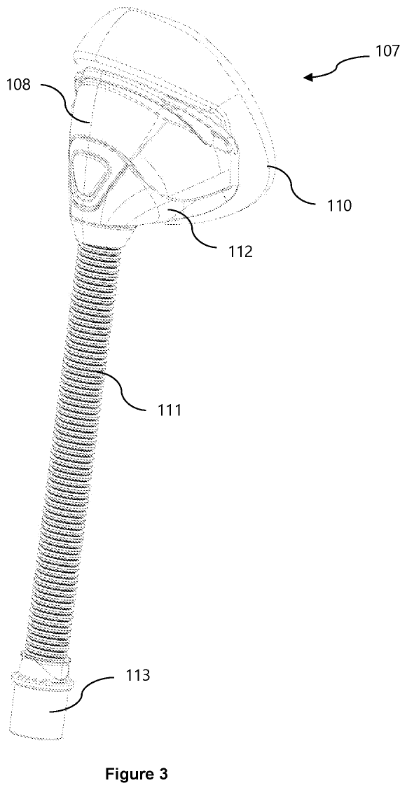

With reference to FIG. 3, patient interface 107 comprises a full face patient interface comprising a relatively rigid cushion support 108, a relatively soft seal 110 that seals against the face of the patient. The cushion support 108 is mounted on a relatively rigid frame 112 to which the headgear (not shown) is connected. Upper end of first gas delivery tube 111 is connected to the patient interface 107. The lower end of the first gas delivery tube 111 comprises, or is connected to, a swivel connector 113, as will be described below.

As used herein, ‘gas delivery tube’ is a broad term and is to be given its ordinary and customary meaning to a person of ordinary skill in the art (that is, it is not to be limited to a special or customized meaning) and includes, without limitation, cylindrical and non-cylindrical elongate gas flow components defining a gases flow path through the component. A gas delivery tube comprises a lumen or a passageway, through which gases can flow. A gas delivery tube comprises a hollow, elongate body that is configured for use in providing respiratory therapy, and that otherwise meet applicable standards for such use.

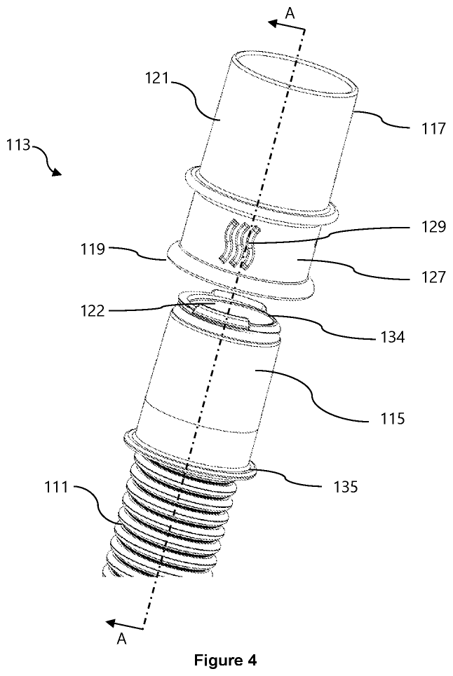

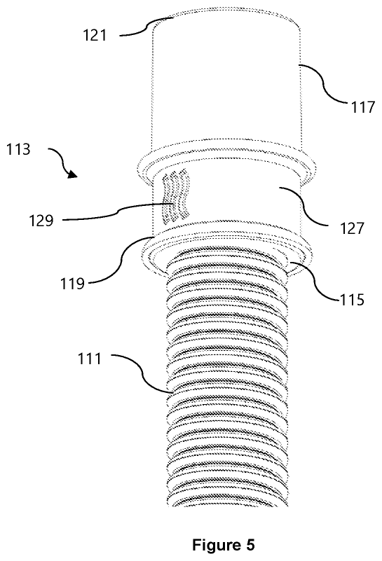

With reference to FIGS. 4 to 5, a swivel connector 113 forms a pneumatic connection, i.e., a gases flow path, between the breathing tube 109 and the connecting tube 111.

The swivel connector 113 comprises a first conduit component 115 and second conduit component 117 that are configured to be connected together.

The first conduit component 115 is connected to the connecting tube 111 and the second conduit component 117 is configured to connect to the breathing tube 109.

The first conduit component 115 and second conduit component 117 are cylindrical and arranged co-axially, with the second conduit component 117 being external to the first conduit component 115. In other words, the first conduit component 115 is an internal component, and the second conduit component 117 is an external component. The first conduit component 115 is a male conduit component and the second conduit component 117 is a female conduit component.

The internal component and 115 and external component 117 share a longitudinal axis 114.

The swivel connector 113 allows relative rotation between the internal component 115 and external component 117, and therefore relative rotation between the breathing tube 109 and connecting tube 111.

The external component 117 and internal component 115 can be formed from a rigid or semi-rigid material, for example a plastics material such as polycarbonate or polypropylene.

The external component 117 comprises a first, proximal end 119 and second, distal end 121.

The distal end 121 is configured to connect to the end of the breathing tube 109, for example with an intermediate coupling component (not shown).

The proximal end 119 of the external component 117 is configured to receive the internal component 115.

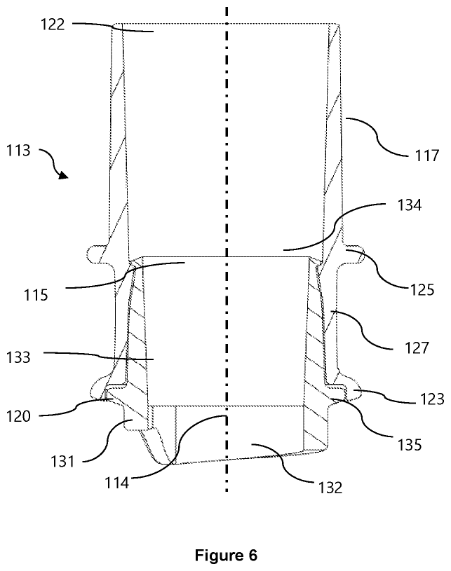

FIG. 6 illustrates a cross-sectional view of the swivel connector of FIGS. 4 and 5. The internal component 115 comprises a first, proximal end 131 and a second, distal end 133 corresponding to the proximal end 123 and distal end 125 of the external component 117. The distal end 133 of the internal component 115 is configured to be received by the proximal end 123 of the external component 117 and is configured to connect to the connecting tube 111 (not shown in FIG. 4).

The first conduit component, or internal component 115 may comprise a proximal opening 132 at the proximal end 131, and a distal opening 134 at the distal end 133, a wall extending between the openings in a direction of the longitudinal axis 114 of the first conduit component 115. The wall may comprise an exterior surface 148 of the first conduit component 115, extending in a direction of a longitudinal axis 114 of the first conduit component 115.

The proximal opening 132 may comprise an inlet to a gases flow path through the first conduit component 115.

The distal opening 134 may comprise an outlet of the gases flow path through the first conduit component 115.

The second conduit component, or external component 117 may comprise a proximal opening 120 at the proximal end 119, the proximal opening 120 comprising an inlet, and a distal opening 122 at the distal end 121, the distal opening 122 comprising an outlet. A wall extends between the inlet and the outlet in a direction of a longitudinal axis 114 of the second conduit component 117. The wall may comprise an interior surface 150 of the second conduit component 117, extending in a direction of a longitudinal axis 114 of the second conduit component 117.

The exterior surface 148 may be substantially aligned with the interior surface 150. The exterior surface 148 may be parallel with the interior surface 150. One surface may be inclined relative to the other surface.

The distal end 121 of the external component 117 is configured to non-rotatably connect to the end of the breathing tube 109, via friction or interference fit. In some examples, the distal end 121 of the external component 117 is received by the breathing tube 109. The non-rotatable coupling enables the user to break the connection between the external component 117 and breathing tube 109 by applying a twisting motion or a torque to the external component 117, before removing the swivel connector 113.

The external component 117 may comprise an external shoulder or flange 123 to which a user may apply an axial pushing or pulling force when connecting or removing the external component 117 to/from the breathing tube 109. The shoulder 123 may be provided towards the proximal end 119 of the external component 117.

The shoulder 123 is an annular ring that protrudes from the exterior surface of the external component 117 that extends fully, or at least partially around the periphery of the external component 117.

In some embodiments, there may be one or more slots or gaps dividing the shoulder 123 into two or more discrete sections arranged peripherally around the external component 117.

In some embodiments, the external component 117 comprises a second, distal shoulder or flange 125 that is closer to the distal end 121 of the external component 117 than the more proximal shoulder 123. The user may apply an axial pushing or pulling force to the proximal or distal shoulders 123, 125 when connecting or removing the external component 117117 to/from the breathing tube 109.

The distal shoulder 125 may also act as end-stop to the breathing tube 109 and prevent the external component 117 from being inserted too far into the breathing tube 109.

In some embodiments, the external component 117 comprises a gripping region 127 provided between the proximal and distal shoulders 123, 125. The gripping region 127 is provided for the user to place one or more of their fingers and apply torque to the external component 117 while connecting or removing the external component 117 to/from the breathing tube 109. The proximal and distal shoulders 123, 125 protrude radially outwardly relative to the surface of the gripping region 127.

The gripping region 127 may comprise gripping features to help provide a secure connection between the user's hand and the external component 117. The gripping features can be in the form of protrusions, ridges, indentations, and/or a textured surface. As illustrated in FIG. 3, the gripping region 127 may comprise one or more protrusions 129, such as elongate ridges, protruding from the exterior surface of the gripping region 127.

The proximal end 131 of the internal component 115 is non-rotatably coupled to the connecting tube 111 via overmoulding. The interior and exterior walls of the connecting tube 111 are surrounded by the internal component 115 such that the lumens of the internal component 115 and the connecting tube 111 are continuous.

As can be seen in FIG. 6, the internal component 115 comprises a shoulder 135. When the internal component 115 and external component 117 are assembled, the shoulder 135 abuts the proximal end 123 of the external component 117. This functions as an end stop, limiting the distance the internal component 115 can be inserted into the external component 117. This also forms a more tortuous, non-linear path, reducing the amount or likelihood of dirt build up in the space or gap formed between the interior surface of the external component 117 and exterior surface of the internal component 115.

The shoulder 135 is located part way along the length of the internal component 115 in the axial direction such that the proximal end 131 is not fully covered by the external component 117. That is, the proximal end 131 is not enclosed by the external component 117 and is not concealed from the user.

The axial direction refers to the direction coinciding with the central longitudinal axes of the cylindrical internal and external components 115, 117, i.e., the axis or direction extending between the proximal and distal ends of the internal and external components 115, 117.

With reference to the cross-sectional view illustrated in FIG. 6, the proximal shoulder 123 of the external component 117 has an inverted L-shape. In other words, the proximal shoulder 123 has a first portion protruding radially and perpendicularly from the exterior surface 152 of the external component 117 away from the longitudinal axis 114, and a second portion that extends in a proximal direction, aligned with the longitudinal axis 114. Thus, the first portion is adjacent the distal surface of the shoulder of the internal component 115 and the second portion is adjacent the radial surface 154 of the shoulder 135 of the internal component 115. This forms an L-shaped tortuous path between the internal and external components 115, 117.

In some embodiments, the proximal shoulder 135 of the internal component 115 is generally co-planar with the proximal shoulder 123 of the external component 117. In other words, the proximal shoulders 135, 123 are disposed at similar locations along the longitudinal axis 114.

The internal component 115 is rotatably coupled to the external component 115 and non-rotatably coupled to the connecting tube 111, enabling the connecting tube 111 to rotate or swivel independently from the external component 117 and therefore the breathing tube 109. This can decrease the likelihood of the connecting tube 111 becoming tangled or twisted in response to patient movement. Thus, the internal component 115 can move with relation to patient movement and can account for the patient's movement. This may increase patient comfort and acceptance of the respiratory therapy.

The exterior surface 148 of the internal component 115 may have a diameter of between approximately 16 mm and 21 mm and the exterior surface 152 of the external component 117 may have a diameter of between approximately 20 mm and 22 mm.

The external component 117 and internal component 115 are connected together by a permanent interference fit, such as by a snap-fit connection.

The interior surface 150 of the external component 117 and exterior surface 148 of the internal component 115 comprise connection features that engage each other to form a snap-fit connection.

The connection features are such that the internal and external components 115, 117 are able to be assembled without requiring a significant amount of axial force (i.e., able to be assembled by hand) but cannot subsequently be disconnected by hand (i.e., form a permanent connection). After assembly, the internal and external components 115, 117 are able to rotate independently from, and relative to, each other, whilst maintaining a secure connection.

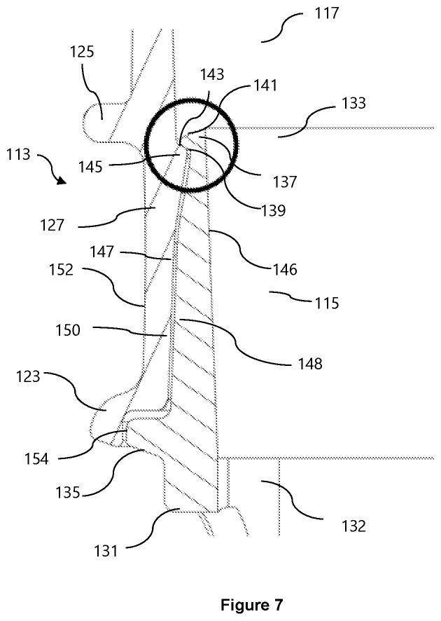

With reference to FIG. 7, the exterior surface 148 of the internal component 115 has an engagement formation in the form of a protrusion 137 extending peripherally around its distal end 131. The internal component 115 tapers towards the distal end 131 such that there is an undercut 139 extending radially, adjacent the protrusion 137. In other words, the protrusion 137 extends over the undercut 139.

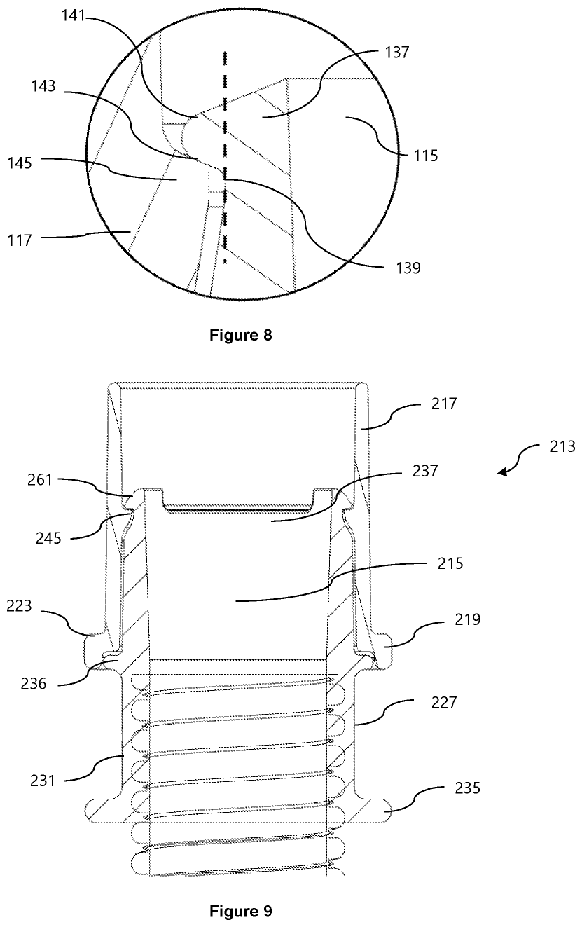

When viewed in cross-section, the protrusion 137 of the internal component 115 has a wedge shape with a sloped distal surface 141 acting as a lead-in to facilitate insertion of the internal component 115 into the external component and a sloped proximal surface 143 that engages with the external component 115. As shown in FIG. 8, a cross-sectional view of the protrusion 137, the proximal surface 143 has an angle of approximately 65 degrees with reference to the axial or longitudinal direction (illustrated as a vertical line in FIG. 8). This acts as the retraction side of protrusion 137. The distal surface 141 has an angle of approximately 45 degrees with reference to the axial direction (vertical in illustration). This acts as the entrance side of protrusion 137. The angle of the proximal surface 143 contributes to the interference fit between the internal component 115 and external component 117, and therefore the permanent snap-fit connection. The angle of the distal surface 141 contributes to the ease of insertion of the internal component 115 into external component 117 during assembly.

A ledge 145 is disposed on the interior surface 150 of the external component 117 such that it may engage with the protrusion 137 on the distal end 131 of the internal component 115 to enable a snap-fit connection between the internal and external components 115, 117. The protrusion 137 of the internal component 115 has a greater external diameter than the ledge 145 of the external component 117 such that there is interference between the two components, providing a permanent snap-fit connection.

The proximal surface 143 of the protrusion 137 of the internal component 115 abuts the ledge 145 of the external component when the two components are assembled such that the internal component 115 and external component 117 are rotatably connected. The ledge 145 may provide a bearing surface for the protrusion 137 wherein the proximal surface 143 of the protrusion 137 can contact the ledge 145 in use. The contact between the ledge 145 of the external component 117 and protrusion 137 of the internal component 115 may also reduce leakage of gases.

The distal end 131 of the internal component 115 extends into the internal cavity of the external component 117 such that the internal component 115 terminates part way along its length in the axial direction. The ledge 145 of the external component 117 is thus located part way along the length of internal component 115.

The external component 117 is connected to the internal component 115 such that the user cannot grip the internal component 115. In other words, the external component 117 surrounds or covers a substantial length of the internal component 115, such that the external component 117 encloses the internal component 115, such that the internal component 115 is covered.

The internal component 115 is received inside the external component 117 such that the internal component 115 does not project sufficiently to be gripped by the user. Any part of the internal component 115 that is exposed, may be insufficient to be gripped by a user, or at least may be insufficient to enable a user to grip the internal component 115 with sufficient force to the internal component 115 to damage and/or break the connection between the internal and external conduit components, 115, 117.

The portion of the proximal end 131 of the internal component 115 that is exposed may be less than 10 mm. The portion that is not covered by the external comment is 117 may be approximately 6 mm.

This can encourage the user to grip the gripping region 127 of the external component 117, to minimise the ability for the user to pull on the internal component 115, as this may cause mechanical stress on the snap-fit features connecting the internal component 115 to the external component 117 (e.g., protrusion 137).

A relatively high axial force may be required to remove and/or separate the swivel connector 113 from respiratory components and may contribute to tearing or damage to the connecting tube 111 and/or the breathing tube 109. Therefore, an indicator for the intended location for gripping the swivel connector 113 increases usability but also reduces the risk of damage to the swivel connector 113 and breathing tubes that are part of the respiratory therapy system.

In some embodiments, the length between the shoulder 135 and the distal end 133 of the internal component 115 is greater than the length between the shoulder 135 and the edge of the proximal end 131 of the internal component 115, wherein the edge of the proximal end delimits the internal component 115 in an axial direction. The former length may be approximately 13 mm and the latter length may be approximately 6 mm. The length between the shoulder 135 and the distal end 133 of the internal component 115 may therefore be more than 1.5 times the length between the shoulder 135 and the edge of the proximal end 131 of the internal component 115. In some embodiments, the length between the shoulder 135 and the distal end 133 of the internal component 115 may be more than 2 times the length between the shoulder 135 and the edge of the proximal end 131 of the internal component 115.

After assembly, that is, when the first and second components 115, 117 are permanently connected together, a clearance 147 is provided between the internal component 115 and the external component 117. This facilitates rotatable connection between the internal component 115 and external component 117 as the external surface 148 of the internal component 115 and internal surface 150 of the external component 117 are not in full contact along their length, while being sufficiently close to provide for insignificant or negligible leakage of gases between the two components 115, 117.

The clearance 147 between the internal component 115 and external component 117 can allow for easier cleaning of the two components as they are unable to be disassembled after engagement of the permanent snap-fit connection.

Referring again to FIG. 7, the distance between the internal surfaces of the external component 117 and the external surfaces of the internal component 115 (i.e., clearance 147) is not constant along the axial direction of the two components. There is a greater radial clearance at the location of the undercut 139 than near the distal end 131 of the internal component 115. This allows for dirt trapped between the internal and external components at this location to be more easily flushed and cleaned under water. Clearance 147 may have a radius of between approximately 0.15 and 0.25 mm, for example approximately 0.18 mm at the location of the undercut 139, and between approximately 0.12 mm and 0.14 mm along the axial length of the internal component 115.

The thickness of the walls of the internal and external components 115, 117 formed between their respective interior and exterior surfaces, 146, 148 and 150, 152, vary in the axial direction. The walls of the internal and external components 115, 117 taper to a smaller thickness at the respective distal ends 121, 131. This allows for the components 115, 117 to be removed from a mould tool in embodiments in which the components are injection moulded.

The axial length of the external component 117 is such there is a sufficient contact between the external component 117 and the breathing tube 109, thus allowing a secure connection. However, a length that is too long may affect the flexibility of the breathing tube system. The length of the external component 117 may be between approximately 30 and 40 mm, and for example approximately 36 mm.

The axial length of the internal component 115 is such that there is sufficient overlap between the internal component 115 and external component 117 when the swivel connector 113 is assembled, allowing a secure connection. A length that is too long may lead to an increased length of the clearance 147, thus increasing the likelihood of dirt building up in the clearance 147. The length of the internal component 115 may be between 10 and 30 mm, and for example approximately 20 mm.

The overlap in axial length between the internal component 115 and external component when the swivel connector 113 is connected is approximately 14 mm. In other words, the internal component 115 extends to approximately 14 mm along the axial length of the external component 117, measured from the proximal end 119. There is an overlap of at least 25% of the axial length of the internal component 115 and external component 117 when the swivel connector 113 is connected, and in some embodiments, the overlap is approximately 40%. When connected, the swivel connector 113 may have an overall length of between 30 and 50 mm, for example approximately 43 mm.

In some embodiments, the external component 117 has a different material appearance to the internal component 115. For example, the external component 117 may have an opaque finish and the internal component 115 may have a translucent (e.g., transparent, or semi-transparent) finish. The opaque finish of the external component 117 reduces the visibility of the internal component 115 and discourages the handling of the internal component 115. The translucent finish of the internal component 115 further reduces its visibility, as it may be of a similar finish or appearance to the connecting tube 111.

FIG. 9 to FIG. 14 illustrate swivel connectors with similar features to the swivel described above with reference to FIGS. 4 to 8.

The swivel connectors 213, 313, 413, 513, 613 of FIGS. 9 to 14 comprise an internal component 215, 315, 415, 515, 615 and external component 217, 317, 417, 517, 617. The internal component 215, 315, 415, 515, 615 is coupled to the connecting tube 111 and the external component 217, 317, 417, 517, 617 is configured to couple to the breathing tube 109. The internal component 215, 315, 415, 515, 615 and external component 217, 317, 417, 517, 617 are cylindrical and arranged co-axially, with the external component 217, 317, 417, 517, 617 being external to the internal component 215, 315, 415, 515, 615. The internal component 215, 315, 415, 515, 615 has an engagement formation in the form of a protrusion that engages with a ledge on an interior surface of the external component 217, 317, 417, 517, 617, providing a snap-fit connection between the two components.

With reference to FIG. 9, an embodiment of a swivel connector 213 comprises a proximal end 231, of which a portion is exposed and extends beyond the proximal end 219 of the external component 217. The internal component 215 comprises two shoulders, a proximal shoulder 235 and distal shoulder 236 both protruding radially from the exterior surface. Between the proximal and radial shoulders 235, 236 there is a gripping region 227 configured to be gripped by the user. The external component comprises a single proximal shoulder 223 located at or adjacent the proximal end that can also be gripped in use.

As with the swivel connector 113 of FIGS. 4 to 8, the distal end of the internal component 215 comprises a radial protrusion 237 configured to engage with a corresponding ledge 245 on the internal surface of the external component 217. Swivel connector 213 further comprises diametrically opposed flexing arms 261 that extend further in the axial direction. As with the radial protrusion 237 of the internal component 215, the flexing arms 261 extend over an undercut. Due to a greater length while having a similar radial overhang, the flexing arms 261 are more rigid and resistant to flexing than the radial protrusion 237. This provides a stronger snap-fit connection.

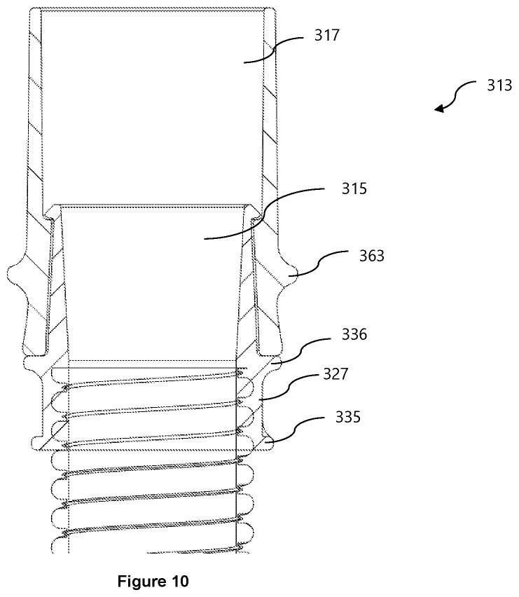

With reference to FIG. 10, swivel connector 313 comprises a portion of the internal component 315 which is exposed and extends beyond the proximal end of the external component 317. The internal component 315 comprises two shoulders, a proximal shoulder 335 and distal shoulder 336, both protruding radially outwardly from the exterior surface of the internal component 315. Between the proximal and radial shoulders 335, 336 there is a gripping region 327 configured to be gripped by the user. The external component 317 has an annular protrusion 363, part way along its length in the axial direction, further adding a location for the user to grip when handling the swivel connector 313.

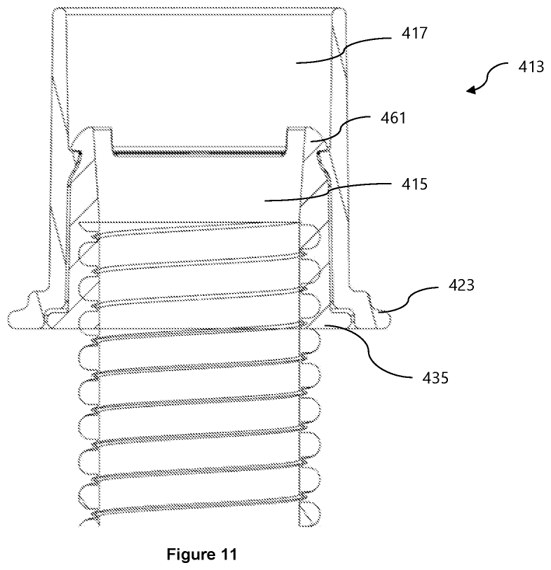

With reference to FIG. 11, swivel connector 413 comprises external component 417 which covers a substantial length of the internal component 415. The internal component 415 has a shoulder 435 disposed towards the proximal end and the external component 417 has a shoulder 423 disposed towards the proximal end. The shoulder 435 of the internal component 415 and shoulder 423 of the external component 417 abut each other, the shoulder 435 of the internal component 415 acting as an end-stop and limiting the further insertion of the internal component 415 into the external component 417. The shoulder 423 of the external component 417 covers the distal surface and outer radial surface of the shoulder 435 of the internal component 415, as seen in the swivel connector of FIGS. 4 to 8. The shoulder 423 of swivel connector 413 protrudes further radially, forming a wider shoulder for the user to grip. As seen in the swivel connector illustrated in FIG. 9, the swivel connector 413 also comprises an internal component 415 with flexing arms 461.

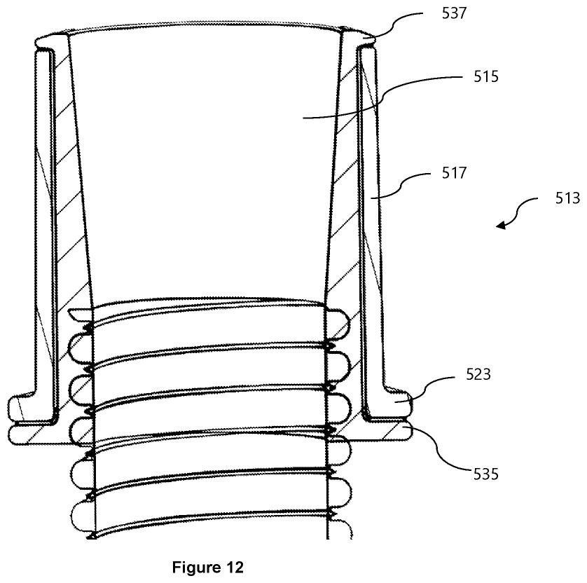

With reference to FIG. 12, swivel connector 513 comprises an external component 517 which covers a substantial length of the internal component 515 such that it cannot be gripped by the user. The internal component 515 has a shoulder 535 at the proximal end and the external component 517 also has a shoulder 523 disposed towards the proximal end. When assembled, the shoulders of the two components abut each other. The distal end of the internal component 515 has a radial protrusion 537 that engages with the distal end 521 of the external component 517 such that the radial protrusion 537 is exposed at the distal end of the external component 517. The internal component 515 and external component 517 may therefore have equal or similar axial lengths. The entire length from the shoulder 535 to the distal of the internal component 515 is contained wholly within the external component 517.

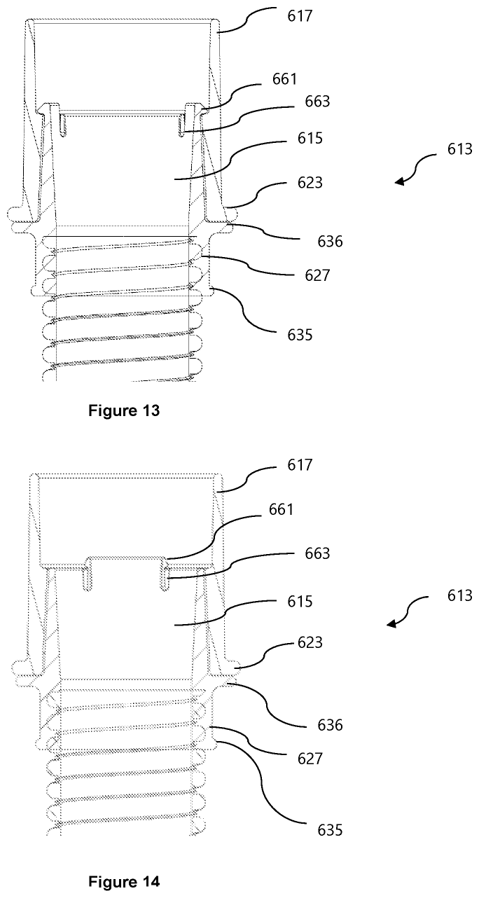

With reference to FIGS. 13 and 14, swivel connector 613 comprises external component 617 which does not cover a substantial length of the internal component 615 and the proximal end of the internal component 615 is exposed such that the user is able to grip it. The internal component 615 has gripping region 627 at the proximal end between a proximal shoulder 635 and distal shoulder 636. The proximal shoulder 635 forms the proximal end 631 of the internal component 615, and the distal shoulder 636 abuts a shoulder 623 on proximal end of the external component 617. As also seen in the swivel connector 213 of FIG. 9, the internal component 615 comprises flexing arms 661. The present embodiment also comprises slots 663 between the flexing arms 661 at the distal end to allow for easier flexure of flexing arms 661.

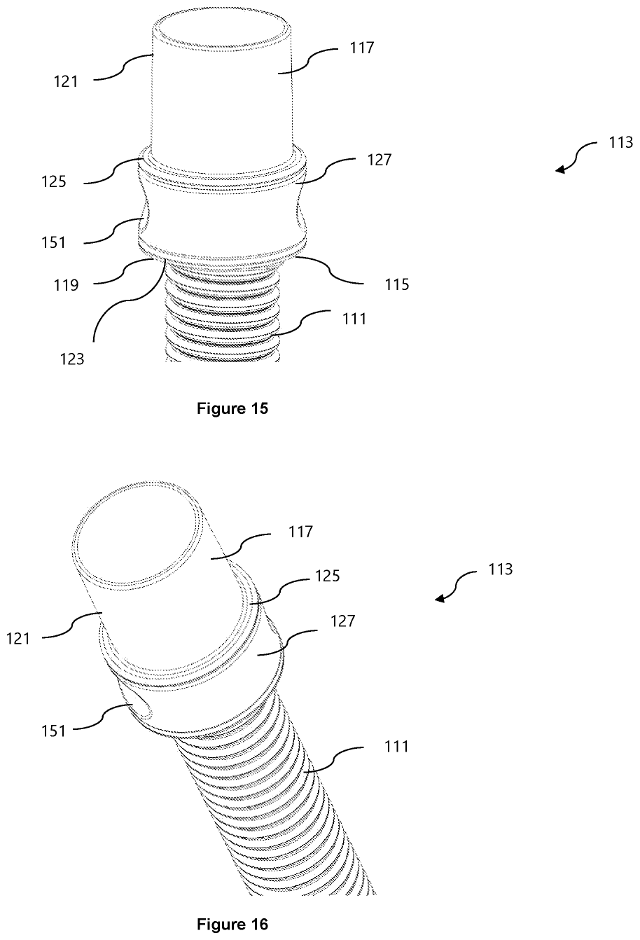

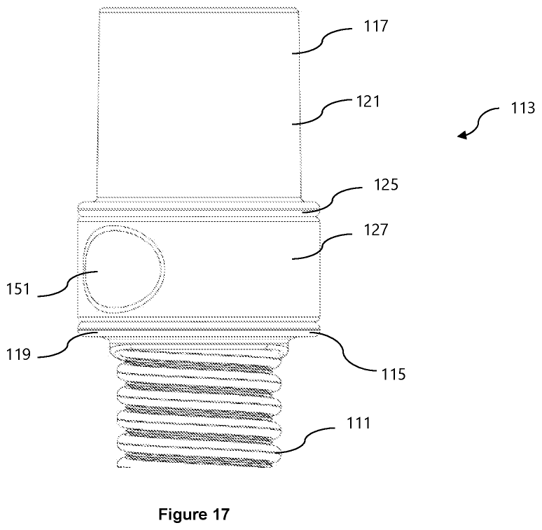

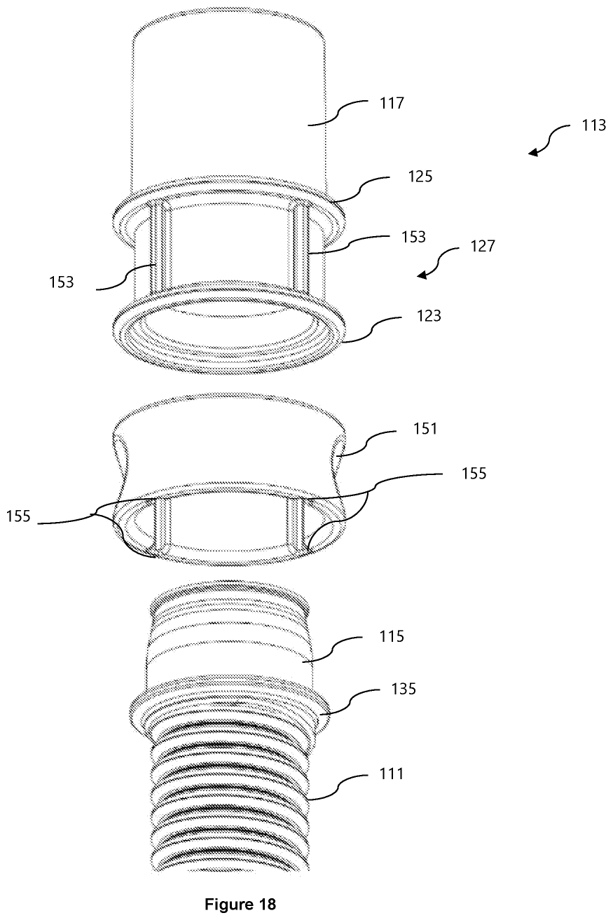

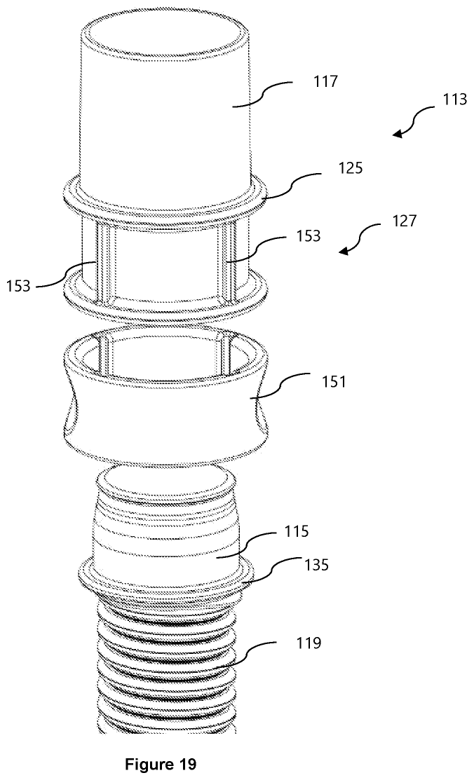

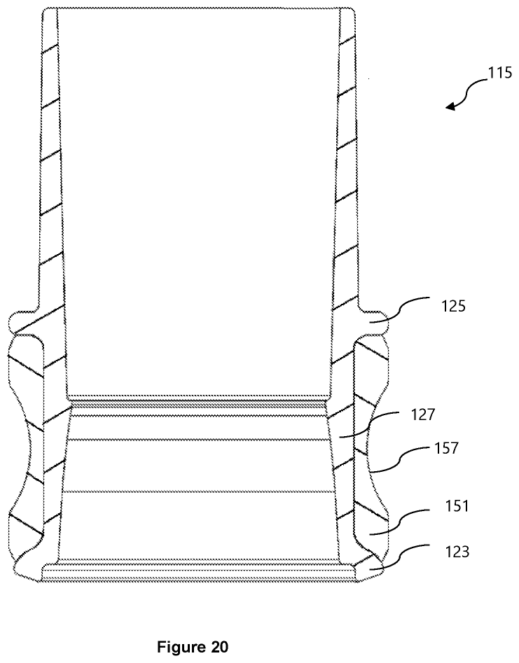

With reference to FIGS. 15 to 19, the external component 117 comprises a gripping region 127 located between the proximal and distal shoulders 123, 125, and a grip ring 151 located at the gripping region. The proximal and distal shoulders 123, 125 limit the axial movement of the grip ring 151 during handling of the gripping region 127 that may occur during connection or removal of the external component 117 from the breathing tube 109.

The grip ring 151 is formed of a high friction material such as silicone and is located on the gripping region 127 of the external component 117. This improves the ability of the user to grip onto the grip ring 151, and therefore the external component 117. In turn, this improves the ability for the user to apply torsion between the external component 117 and the breathing tube 109 during connection and disconnection of the two components.

The gripping region 127 of the external component 117 further comprises ribs 153 protruding radially from the exterior surface configured to engage with corresponding recesses 155 on the interior surface of the grip ring 151. This minimises the relative rotation between the grip ring 151 and the external component 117, thus allows for a more secure grip of the external component 117.

The grip ring 151 is moulded as a separate component from the external component 117. The grip ring 151 is formed of a resilient material, allowing for it to be stretched on to the gripping region 127 of the external component 117 for assembly. A separately moulded grip ring 151 helps with improving tolerance and reducing heat sinks and flashing that may occur during moulding.

The grip ring 151 can be overmoulded onto the gripping region 127 of the external component 117.

The grip ring 151 may be located on the gripping region 127 of the internal component 115, where present.

The grip ring 151 may be a colour that is different to that of the internal or external components 115, 117. This differentiates the grip ring 151 and gripping region 127 from the rest of the swivel connector 113, providing an indication to a user of the location that should be gripped during use.

As shown in FIG. 20, in some embodiments, the grip ring 151 may have grip features on the exterior surface, such as depressed regions 157 to accommodate placement of fingers and improve grip when gripped by the user. In other embodiments, the grip features may be any other embodiments that improve grip such as ridges, protrusions, indentations, or textured surfaces.

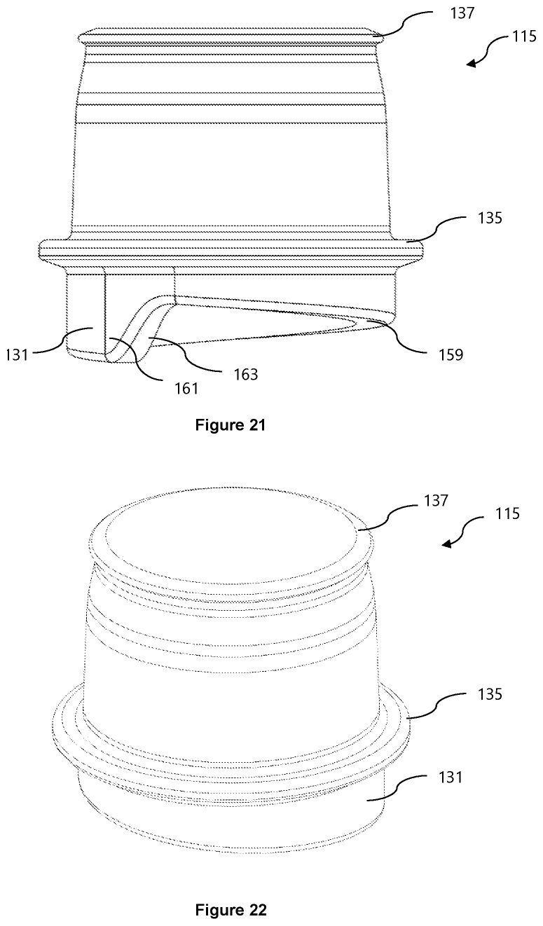

As illustrated in FIGS. 21 and 22, in some embodiments, the proximal end 135 of the internal component 115, which is coupled to the connecting tube 111, comprises an offset portion 161 formed by a step change 163 in the edge 159 of the proximal end 135. The edge 159 delimits the internal component 115 in an axial direction. An offset portion 161 is described in our earlier publication WO2016007019A1, the entire contents of which are hereby incorporated by reference.

The axial length of the proximal end 135 of the internal component 115 varies circumferentially in the embodiments illustrated in FIGS. 21 and 23. The offset portions 161, 167 refer to the portions of the proximal end 135 with the greatest distance in the axial direction (i.e., extending between proximal end 135 and distal end 133) from the distal end 133 of the internal component 115, relative to that of the remaining portions of the proximal end 135.

The offset portion 161 of the internal component 115 is configured to allow the remainder of the edge 159 of the proximal end 135 to follow the helical orientation of the bead of the connecting tube 111. This improves the strength of the overmould bond between the internal component 115 and the connecting tube 111, allowing it to support increased axial load, and/or reducing the likelihood of tearing of the connecting tube 111 at or near the location of the overmould.

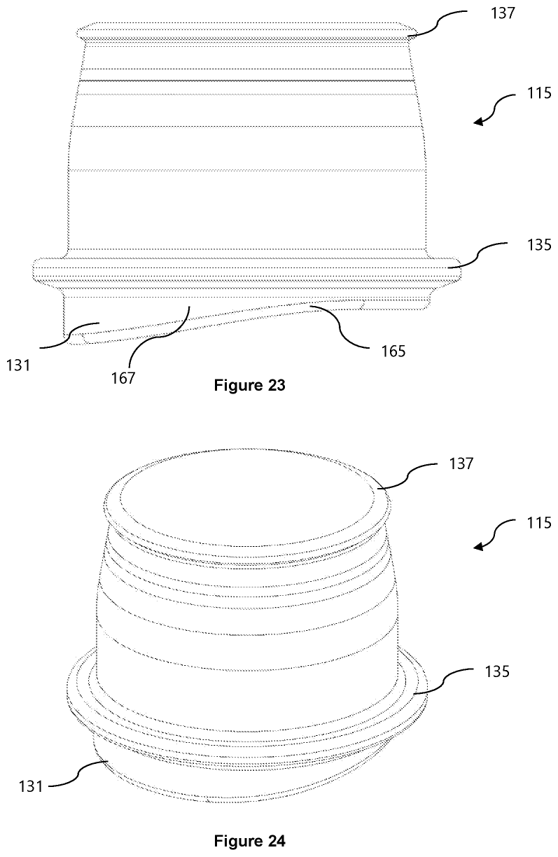

With reference to FIGS. 23 and 24, the proximal end 135 of the internal component 115 has an axial length that varies circumferentially. The edge 165 of the proximal end 135 comprises a continuous, smooth curve providing a transition to and from the offset portion 167. This may further strengthen the overmould bond between the internal component 115 and the connecting tube 111 as the stresses experienced at the location of the overmould are more radially consistent. As seen in the side view illustrated in FIG. 23, the offset portion 167 diametrically opposes a section of the proximal end 135 with a constant axial length.

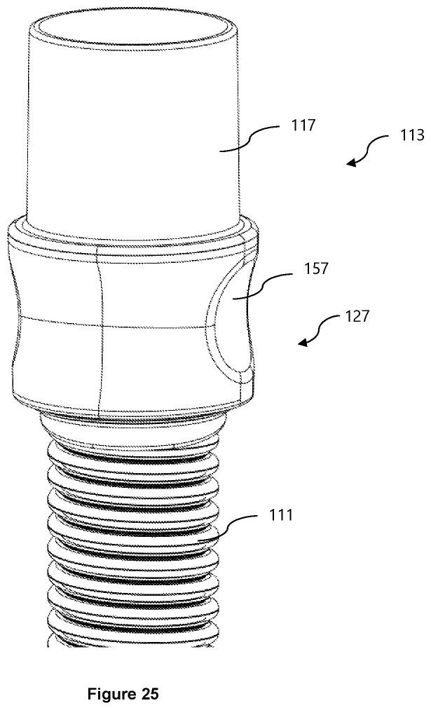

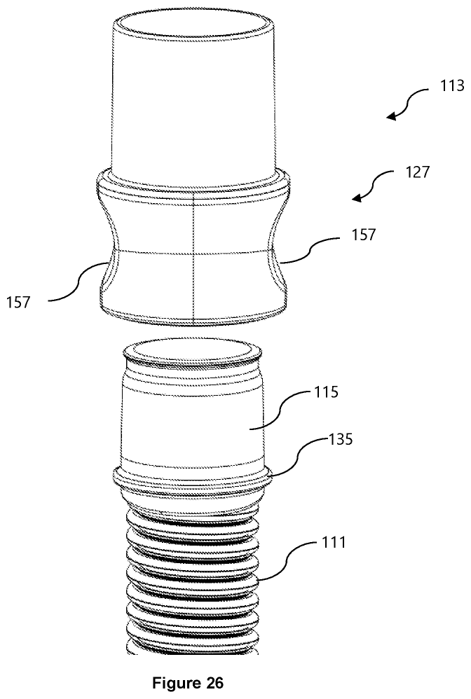

With reference to FIGS. 25 and 26, a swivel connector comprises similar features to those of the swivel connector of FIGS. 22 to 24. However, the external component 117 comprises an integrally formed gripping region 127 formed by the shape and profile of an enlarged diameter portion of the external component 117 itself. The shoulders 123, 125, and the grip ring 151 are not provided. The gripping region 127 comprises a generally concave ring shaped region of the external component 117, the concave shape in itself facilitating gripping by the user. The gripping region further comprises a pair of diametrically opposed depressed regions 157 that further facilitate gripping by the user.

It should be noted that various changes and modifications to the presently preferred embodiments described herein will be apparent to those skilled in the art. Such changes and modifications may be made without departing from the spirit and scope of the apparatus and systems of the disclosure and without diminishing its attendant advantages. For instance, various components may be repositioned as desired. It is therefore intended that such changes and modifications be included within the scope of the apparatus and systems of the disclosure. Moreover, not all of the features, aspects and advantages are necessarily required to practice the present apparatus and systems of the disclosure. Accordingly, the scope of the present apparatus and systems of the disclosure is intended to be defined only by the claims that follow.

Reference to any prior art in this specification is not, and should not be taken as, an acknowledgement or any form of suggestion that that prior art forms part of the common general knowledge in the field of endeavour in any country in the world.

Wherein the foregoing description reference has been made to integers or components having known equivalents thereof, those integers are herein incorporated as if individually set forth.

Unless the context clearly requires otherwise, throughout the description and the claims, the words “comprise”, “comprising”, and the like, are to be construed in an inclusive sense as opposed to an exclusive or exhaustive sense.

The apparatus and system of the disclosure may also be said broadly to consist in the parts, elements and features referred to or indicated in the specification of the application, individually or collectively, in any or all combinations of two or more of said parts, elements or features.

1-68. (canceled)

69. A swivel connector for a gas delivery tube of a respiratory therapy system, the swivel connector comprising:
a first conduit component; and
a second conduit component configured to receive the first conduit component;
wherein the first conduit component comprises a proximal end configured to be connected to the gas delivery tube, and a distal end;
wherein the first conduit component and the second conduit component are configured to be permanently connected together, and to be rotatable relative to one another, when connected together;
wherein the first conduit component is configured to be coupled to the gas delivery tube via overmoulding.

70. The swivel connector of claim 69, wherein an exterior surface of the first conduit component is adjacent an interior surface of the second conduit component.

71. The swivel connector of claim 69, wherein the first conduit component and the second conduit component are arranged coaxially.

72. The swivel connector of claim 69, wherein the first conduit component and the second conduit component are connected by a snap-fit mechanism.

73. The swivel connector of claim 72, wherein the first conduit component comprises at least one engagement formation configured to engage the second conduit component to provide the snap-fit mechanism.

74. The swivel connector of claim 73, wherein the interior surface of the second conduit component comprises at least one radially extending protrusion configured to engage with the at least one engagement formation of the first conduit component.

75. The swivel connector of claim 74, wherein the at least one radially extending protrusion is a ledge.

76. The swivel connector of claim 75, wherein the ledge extends peripherally around the interior surface of the second conduit component.

77. The swivel connector of claim 75, wherein when assembled, the ledge is configured to provide a bearing surface for the at least one engagement formation of the first conduit component.

78. The swivel connector of claim 69, wherein the first conduit component has a length between the proximal end and the distal end along a longitudinal axis, wherein the first conduit component may be configured to be connected to the second conduit component such that at least 25% of the length of the first conduit component is inside the second conduit component.

79. The swivel connector of claim 69, wherein the second conduit component has a length between the proximal end and the distal end along a longitudinal axis, wherein when connected, the first conduit component overlaps approximately 40% of the length of the second conduit component.

80. The swivel connector of claim 69, wherein the first conduit component comprises at least one shoulder extending outwardly at least partially around a perimeter of an exterior surface of the first conduit component.

81. The swivel connector of claim 80, wherein the shoulder is disposed towards the proximal end of the first conduit component.

82. The swivel connector of claim 80, wherein the shoulder is configured to abut a proximal end of the second conduit component.

83. The swivel connector of claim 80, wherein the shoulder of the first conduit component is configured to abut a shoulder of the second conduit component.

84. The swivel connector of claim 69, wherein the first conduit component and the second conduit component are each generally cylindrical.

85. The swivel connector of claim 69, wherein, when the first conduit component is connected to the second conduit component, a portion between the shoulder and the distal end of the first conduit component is covered by the second conduit component, and a portion between the proximal end and the shoulder of the first conduit component is exposed.

86. The swivel connector of claim 85, wherein the portion between the shoulder and the distal end of the first conduit component is enclosed by the second conduit component, such that this portion is covered by the first conduit component.

87. The swivel connector of claim 85, wherein the portion between the shoulder and the distal end of the first conduit component has a first length along a longitudinal axis and the portion between the shoulder and the proximal end of the first conduit component may have a second length along the longitudinal axis, wherein the first length is more than the second length.

88. The swivel connector of claim 69, wherein the proximal end of the first conduit component further comprises an edge that delimits the first conduit component along a longitudinal axis, and the edge comprises an offset portion, the offset portion being configured to allow a portion of the edge to follow a helical orientation of part of the gas delivery tube.

89. The swivel connector of claim 69, wherein the second conduit component comprises a gripping region which preferably comprises a generally concave ring shaped region, the concave shape in itself facilitating gripping by a user.

90. The swivel connector of claim 89, wherein the gripping region comprises a pair of diametrically opposed depressed regions that further facilitate gripping by the user.

91. The swivel connector of claim 69, wherein the swivel connector comprises only two components, namely the first conduit component and the second conduit component that are configured to be connected together; wherein the first conduit component is configured to be connected to a connecting tube and the second conduit component is configured to be connected to a breathing tube.

92. A patient interface assembly comprising:
a patient interface;
a gas delivery tube configured to be connected to the patient interface; and
the swivel connector of claim 69.
